(19) United States
(12) Patent Application Publication
(10) Pub. No.: US 2012/0157395 A1
(43) Pub. Date: Jun. 21, 2012

(54) ANTI-INFLAMMATORY AGENT FOR ORAL APPLICATION, AND ANTI-INFLAMMATORY PEPTIDE FOR ORAL APPLICATION

(76) Inventors: Masahisa Ibuki, Osaka (JP); Yoshinori Hasegawa, Osaka (JP); Yoshinori Mine, Ontario (CA)

(21) Appl. No.: 13/383,673

(22) PCT Filed: May 12, 2010
(86) PCT No.: PCT/JP2010/057993

§ 371 Date: Mar. 6, 2012

(30) Foreign Application Priority Data
Jul. 13, 2009 (JP) 2009-164321
Jul. 13, 2009 (JP) 2009-164323

(51) Int. Cl. A61K 38/06; A61P 29/00; C07K 5/083; C07K 2/00; C07K 5/087

(52) U.S. Cl. 514/21.9; 530/300; 530/331

(57) ABSTRACT

Disclosed are a composition having an anti-inflammatory function and a therapeutic method using the composition, both of which are effective for inflammatory diseases such as inflammatory bowel disease. Specifically disclosed are: an anti-inflammatory functional agent for oral application, which comprises a soybean peptide, wherein the soybean peptide contains fractions each having a molecular weight of 500 or less and excluding any free amino acid in an amount of 40 wt % or more and has a free amino acid content of 7 wt % or less; and a therapeutic method using the anti-inflammatory functional agent. More specifically disclosed are: a novel tripeptide which comprises the following amino acid sequence: phenylalanine-leucine-valine or valine-proline-tyrosine and has an anti-inflammatory function; an anti-inflammatory composition for oral application, a medicinal agent and a feed, each of which contains the novel tripeptide as an active ingredient; and a method for treating inflammatory bowel disease using the novel peptide.

### TECHNICAL FIELD

The present invention relates to peptides having an anti-inflammatory function. The present invention also relates to novel tripeptides, Phe-Leu-Val and Val-Pro-Tyr, having an anti-inflammatory function.

### BACKGROUND ART

Patent Document 1 is an application pertaining to a peptide which states an anti-inflammatory function. The application describes a great variety of kinds of di- and tripeptides containing Arg and also describes a number of physiological effects in addition to the anti-inflammatory function. However, these are premised on synthetic production and it is difficult to say that the safety is sufficiently established. In addition, there are no descriptions of Phe-Leu-Val and Val-Pro-Tyr.

Patent Document 2 is an application which does not directly state the anti-inflammatory function but states a component derived from foods, which component has an effect on a gastrointestinal ulcer that the effect is also confirmed in the present invention. However, this describes only an effect of enzyme degradation products of cheese, and there are no descriptions of a specific active substance. Also, cheese is more likely to be picky eaters because of its unique flavor, and there is a high possibility that it comes to have a stronger flavor by the enzyme degradation. Further, dairy proteins are proteins which easily cause bitterness by enzyme degradation. Thus, the possibility of use for food materials is restricted.

### PRIOR ART DOCUMENTS

### Patent Documents

Patent Document 1: JP 2001-500492 A
Patent Document 2: JP 2009-120519 A

### SUMMARY OF THE INVENTION

### Problems to be Solved by the Invention

An object of the present invention is to provide a pharmaceutical composition and a food which have an anti-inflammatory function and which are effective against inflammatory diseases such as inflammatory bowel disease, and a method of treatment using said composition. Another object of the present invention is to provide a material which has an anti-inflammatory function and which is effective against inflammatory diseases such as inflammatory bowel disease, and a method of treatment using said material. It is particularly desirable that the material is able to be used as a versatile food material.

### Means for Solving the Problems

The present inventors have intensively studied in view of the above-described present situation, and have found that a bowel inflammation is inhibited by orally administering a soybean peptide, which is prepared to have certain molecular weight distribution by enzyme degradation, to pigs in which the bowel inflammation has been induced by using dextran sulfate, thereby leading to the present invention.

From more detailed studying, the present inventors have found that a secretion of inflammatory cytokines (TNF-α, IL-6 and IL-17) in plasma of pigs to which said soybean peptide is orally administered is inhibited. That is, they have found that the soybean peptide of the present invention has an anti-secretory function on the inflammatory cytokines and the inflammation is inhibited by the function. When peptides existing in blood plasma of specimens which have achieved the effect are analyzed, tripeptides having an amino acid sequence phenylalanine-leucine-valine or valine-proline-tyrosine are found. And they have found that those which have the amino acid sequences are absorbed via bowel epithelial cells and the like, the immune system is stimulated, and thereby expressing the above-described anti-secretory function on anti-inflammatory cytokines. That is, they have found that the tripeptides having the amino acid sequence, phenylalanine-leucine-valine or valine-proline-tyrosine, can be expected as effective materials to alleviate inflammatory bowel disease and the like. The present invention has been completed on the basis of these findings.

The present invention relates to soybean peptides which have certain molecular weight distribution and which have an anti-inflammatory function, and oral anti-inflammatory compositions, pharmaceutical products, foods and feedstuffs which contain said peptides as an active ingredient, as well as methods of treating inflammatory diseases using said peptides. The present invention also relates to novel tripeptides which have an anti-inflammatory function and consist of the amino acid sequence, phenylalanine-leucine-valine or valine-proline-tyrosine, and oral anti-inflammatory compositions, pharmaceutical products and feedstuffs which contain said tripeptides as an active ingredient, as well as methods of treating inflammatory diseases using said tripeptides and the like.

That is, the present invention is:

(1) an oral anti-inflammatory functional agent comprising a soybean peptide, wherein the soybean peptide comprises 40% by weight or more of fractions having a molecular weight of 500 or less except for free amino acids, and wherein the soybean peptide comprises 7% by weight or less of the free amino acids;

(2) a method of treating an inflammation, which comprises using the anti-inflammatory functional agent according to (1);

(3) the oral anti-inflammatory functional agent according to (1), wherein an intended disease of the agent is bowel inflammation;

(4) a tripeptide which has an amino acid sequence Phe-Leu-Val;

(5) a tripeptide which has an amino acid sequence Val-Pro-Tyr;

(6) an anti-inflammatory functional agent comprising one or more of the tripeptides according to (4) or (5) as an active ingredient;

(7) a method of treating an inflammation, which comprises using one or more of the tripeptides according to (4) or (5) as an active ingredient; and

(8) an anti-secretory agent of inflammatory cytokines comprising one or more of the tripeptides according to (4) or (5) as an active ingredient.

### Effect of the Invention

According to the present invention, peptides which are effective against inflammatory diseases such as inflammatory bowel disease, and foods, medicines and feedstuffs containing said peptides are provided. According to the present invention, anti-inflammatory compositions which are effective against inflammatory diseases such as inflammatory bowel disease, and foods, medicines and feedstuffs containing said composition are also provided. Further, methods of treating inflammation using said tripeptides are provided.

### BEST MODE FOR CARRYING OUT THE INVENTION

In the molecular weight distribution, the soybean peptides of the present invention are necessary to contain 40% by weight or more of fractions having a molecular weight of 500 or less (except for free amino acids) and 7% by weight or less of the free amino acids. It is more preferable that the peptides contain 43% by weight or more of fractions having a molecular weight of 500 or less (except for free amino acids) and 6% by weight or less of the free amino acids. The molecular weight herein is measured by a gel filtration method. More specifically, the molecular weight is measured by using a gel filtration column manufactured by TSK and a UV detector. More specifically, the measurement of the molecular weight is carried out by the following method.

Method of Measuring a Molecular Weight

A gel filtration system for peptides is set by series connection of two kinds of columns. Known peptides as molecular-weight marker are charged into the system, and calibration curve of the relationship between molecular weight and retention time is made (Table 1, FIG. 1). An enzyme degraded product (1%) is centrifuged at 10,000×g for 10 minutes and the obtained supernatant is 2-fold diluted using a solvent for gel filtration, and then 5 of the diluted solution is applied to the system. The content ratio (%) of each molecular weight fraction is calculated using the proportion of the area of a specified molecular weight range (time range) to the whole area of the absorbance chart. (1st column: Superdex 75 10/300 GL, 2nd column: Superdex Peptide 7.5/300 GL, solvent: 1% SDS/10 mM phosphate buffer solution, pH 8.0, 25° C., flow rate: 0.25 ml/min, detection: OD 220 nm)

TABLE 1
Molecular weight standard substances

| | | Molec- |
|---|---|---|
| Substance | | ular |
| name | Sequence | weight |
| Neurotensin | | 1672.9 |
| [β-Asp]- | β-Asp-Arg-Val-Tyr-Ile-His- | 1046 |
| Angiotensin | Pro-Phe | |
| II | | |
| Angiotensin | Val-Tyr-Ile-His-Pro-Phe | 774.91 |
| IV | | |
| | Arg-Arg-Gly-Asp-Met-Glu | 762.85 |
| Leu- | Tyr-Gly-Gly-Phe-Leu | 555 |
| Enkephalin | | |
| | Glu-Glu-Glu | 405.36 |
| | Arg-Gly-Asp | 346.34 |
| | Glu-Glu | 276.25 |
| | (Gly)4 | 246 |
| | Leu-Gly-Gly | 245 |
| | Gly-Gly-Gly | 189 |
| | Pro | 115.13 |

In addition, free amino acid content is analyzed by the following method.

Measurement of Free Amino Acid Content

A sample (4 mg/ml) is added to an equal amount of 3% sulfosalicylic acid and the obtained mixture is shaken at room temperature for 15 minutes. The obtained mixture is centrifuged at 10,000 rpm for 10 minutes and the resulting supernatant is filtered with a 0.45 filter, followed by measuring free amino acids by using an amino acid analyzer (JLC500V manufactured by JEOL Ltd.). The amount of amino acids is calculated as an amount relative to the amount of the whole crude protein obtained by the Kjeldahl method.

In the present invention, when the proportion of fractions having a molecular weight of 500 or less is under 40% by weight or the free amino acid content is above 7% by weight in the soybean peptides, the remarkable effect can be lost.

As a method of preparing a soybean peptide which has the above-described molecular weight distribution from a soybean protein, conventionally known methods such as enzyme degradation and acid degradation can be employed. Particularly, the enzyme degradation is more desirable in terms of safety of production workers and the like because the reaction can be carried out under milder conditions.

In degradation of a soybean protein, when degradation is carried out by enzymes i.e. proteases, commercially available products regardless of animal-, plant- or microorganism-origin can be used as the protease.

Specifically, serine proteases (such as trypsin and chymotrypsin derived from animals, and subtilisin and carboxypeptidase derived from microorganisms), thiolpeptidases (such as papain and bromelain derived from plants), carboxyproteases (such as pepsin derived from animals), metal proteases (such as thermolysin) and the like can be used. More concretely, “Thermoase” derived from a microorganism (manufactured by Daiwa Fine Chemicals Co., Ltd.), “Bioprase” (manufactured by Nagase ChemteX Corporation), “Sumizyme FP” (produced by Shin Nihon Chemical Co., Ltd.) and the like can be used. The reaction conditions of the enzymes can be appropriately adjusted in view of mainly an optimal reaction condition of an enzyme to be used and workability and the like. For example, a commercially available soybean protein isolate is used as a substrate, and to an approximately 3 to 7% by weight solution of the protein (pH 5 to 9), approximately 0.5 to 2.0% by weight of an enzyme is added, and reaction is carried out at a temperature between about 30 and 60° C.

To obtain a peptide having the intended molecular weight distribution, some separating steps can also be combined after degradation by enzymes or acids. Specific examples can include removing precipitate produced in degradation step with enzymes and the like by using centrifugation or separation with a UF membrane and the like, and these can be appropriately combined. To efficiently obtain a peptide having the intended molecular weight distribution, use of the separation means is often more advantageous in view of the yield and the like.

“Inflammation” on which the present invention has an effect will now be described. Inflammation means symptoms developed during a process of removing the reasons when cells are damaged by some disorder. The inflammation is caused by removal of viruses and dead cells when having a cold or injuring, and the inflammation is a signal of an attempt to remove the causative substances. Such viruses and bacteria are called exogenous factors. On the contrary, an allergic inflammation caused by deposition of immune complexes produced inside the body in cells and tissues, an inflammation caused by abnormal metabolites generated inside the body (for example, gout) and the like are called endogenous factors.

However, inflammatory diseases of unknown etiology are also found. For example, Inflammatory Bowel Disease (IBD) such as Crohn's disease (CD) and ulcerative colitis (UC) is the example thereof, and it seems that the factors have not yet been identified. A current widely-accepted theory is an abnormality in intestinal immune tolerance, which is believed to be an abnormality in intestinal immune system associated with immunological screening of harmful and harmless substances to the body (immune tolerance) which screening is performed in the intestinal tract. That is, although a body condition does not need to activate the immune system, it is misidentified that harmful substances exist in the intestinal mucosa. As a result, excessive secretion of inflammatory cytokines and excessive migration of leukocytes occur, and uncontrolled inflammation of bodies is caused. Furthermore, even normal cells get damaged.

Although diseases recognized as inflammatory bowel disease are the above-described Crohn's disease and ulcerative colitis, it is predicted that mild diarrhea and the like which are occasionally caused when psychological stress gets stronger also occur by the same mechanism. That is, abnormality in intestinal immune system is caused by psychic and exogenous factors such as stress and then inflammation in the intestinal tract occurs. In fact, there are reports on many cases diagnosed as inflammatory bowel disease in which mild diarrhea starts as an initial symptom and then a severe symptom such as melena appears when stress from work or the like is strongly felt. Further, failures of the intestinal immune system in IBD will be described. In general, so-called naive T cells differentiate into helper T cells such as Th1, Th2 and Th17, which has been recently discovered, by various cytokines.

The helper T cells are important organs responsible for body immune function, and it is known that allergic symptoms such as pollinosis are also caused by failures of the differentiation thereof. It is said that an inflammatory cytokine, TNF-α, secreted from the Th1 cells is deeply involved in CD. In addition, it is believed that Th17 is deeply involved in inflammation and IBD, and inflammatory cytokines responsible for the differentiation and the like are IL-6 and IL-17. As medicines for inflammatory bowel disease, a wide variety of adrenocortical hormones, azathioprine which is an immunosuppressive drug, infliximab having an effect of trapping TNF-α before it binds to a receptor, and the like have shown their effects. This shows that an immunological treatment is effective.

As described above, inflammatory bowel disease and the like have symptoms closely associated with immune mechanism, and it is believed that the quantity can be grasped by measurement of secretory behavior of TNF-α, IL-6, IL-17 and the like. (International J. of Colorectal Disease, 15, 144-160 (2000)). That is, when secretion of TNF-α, IL-6 and IL-17 is inhibited, an anti-inflammatory effect can be confirmed. When the anti-inflammatory effect is high, an anti-inflammatory effect can be determined by measuring absolute values of secretion volumes of TNF-α, IL-6 and IL-17 per tissue weight of a specimen. When the average value of each cytokine is compared to a control, if secretory inhibition of 20% by weight or more is found in any one of TNF-α, IL-6 and IL-17, the anti-inflammatory effect can be considered as “effective”.

Of coarse, when secretory inhibition of 25% by weight or more is confirmed, it can be said that the effect is stronger, and when secretory inhibition of 30% by weight or more is confirmed, it can be said that the effect is further stronger. In addition, even if the secretory inhibition is under 20% by weight, when the value is evaluated as “significant” at a risk rate of 5% or less by the Tukey-Kramer comparison test, an anti-inflammatory effect can be confirmed. In fact, even if an inhibitory amount of each cytokine secretion is low, when the value is evaluated as “significant” by the Tukey-Kramer comparison test, patients often feel an anti-inflammatory action. Thus the evaluation by the Tukey-Kramer comparison test is important.

In the present invention, inflammatory bowel disease is focused among general inflammatory diseases. That is, in the present invention, although inflammatory diseases refer to all diseases associated with general inflammation, the word more specifically refers to inflammatory bowel disease (IBD). Specific examples thereof can include Crohn's disease (CD) and ulcerative colitis (UC). In addition, in mild conditions which are not recognized as diseases, for example, acute diarrhea and abdominal pains as subjective symptoms, as long as the conditions are caused by inflammation, the peptides of the present invention are effective. In this case, these mild conditions are also included in “inflammatory bowel disease” in the present invention.

The peptides of the present invention can be used for foods, medicines and feedstuffs. If necessary, the peptides can be mixed with other raw materials in an appropriate way. When the peptides are supplied as foods, they can be mixed with a solid food such as biscuit, cake and bread, and they can be dissolved in water to be a drink, or can be mixed with fluid and semisolid foods such as yoghurt and pudding. However, the peptides of the present invention can denature, e.g., be degraded by heating during food sterilization. Therefore, it is preferable that the peptides are mixed with vitamins, minerals and the like to be utilized as food supplements. When the peptides are supplied as pharmaceutical products, they can be mixed with other pharmaceutical products such as adrenocortical hormones and immunosuppressive drugs to be used. As feedstuff use, the peptides can be used for animals without being restricted to land animals and marine animals.

The tripeptides of the present invention, having the amino acid sequence, phenylalanine-leucine-valine or valine-proline-tyrosine, can be used for foods, medicines and feedstuffs. If necessary, the tripeptides can be mixed with other raw materials in an appropriate way. An aspect that the tripeptides are supplied as foods is the same as those described for the above-described “peptides”.

The tripeptides of the present invention, having the amino acid sequence, phenylalanine-leucine-valine (abbreviation: Phe-Leu-Val or FLV) or valine-proline-tyrosine (abbreviation: Val-Pro-Tyr or VPY), can be synthesized by a generally known method and can be isolated from a food material. However, those which are isolated from foods may be more advantageous in terms of safety and the like. As an example from foods, in the case of FLV, such an amino acid sequence is present at residues 472 to 474 of the A2B1a subunit of 11S globulin in soybean protein, and can be isolated after degradation of a soybean protein isolate by a protease treatment and the like. Soybean is believed to have high safety on the basis of long history of eating experience. Therefore, when the peptide is isolated from a food material, it is preferable that soybean is a raw material of the food material. Similarly, VPY is present at residues 153 to 155 of the A5A4B3 subunit of 11S globulin in soybean protein.

The tripeptides of the present invention, having the sequence, phenylalanine-leucine-valine or valine-proline-tyrosine, can produce the effect by being taken as peptide mixtures including such tripeptides instead of being isolated to be used. That is, even when the tripeptides of the present invention having the anti-inflammatory action are orally administered, the effect is produced.

When said tripeptides are obtained from soybean proteins, first, the soybean proteins are needed to be degraded by conventionally known methods such as enzyme treatment and acid treatment. Particularly, the enzyme degradation is more desirable in terms of safety of production workers and the like because the reaction can be carried out under milder conditions.

The conditions in which degradation is carried out by enzymes i.e., proteases, are the same as those of the above-described “peptides”.

In the molecular weight distribution, the peptides including the tripeptides having the sequence, phenylalanine-leucine-valine or valine-proline-tyrosine, preferably contain fractions having a molecular weight of 500 or less in an amount of 40% by weight or more (except for free amino acids) and 7% by weight or less of the free amino acids, and more preferably contain fractions having a molecular weight of 500 or less in an amount of 43% by weight or more (except for free amino acids) and 6% by weight or less of the free amino acids. The molecular weight is measured by a gel filtration method. More specifically, the molecular weight is measured by using a gel filtration column manufactured by TSK and a UV detector. When the proportion of fractions having a molecular weight of 500 or less is under 40% by weight or the free amino acid content is above 7% by weight, the content of tripeptides having the sequence, phenylalanine-leucine-valine or valine-proline-tyrosine, can be dramatically lowered.

To obtain the above-described molecular weight distribution of soybean peptides, the soybean proteins are not only degraded with enzymes but can also be subsequently subjected to any separating operation. Specific examples thereof can include removing precipitate produced in the degradation step with enzymes and the like by using centrifugation or separation with a UF membrane and the like. To efficiently obtain the peptides having the intended molecular weight distribution, use of the separation means is often more advantageous in view of the yield and the like.

As methods of separating such tripeptides, conventionally known methods can be used. For example, one or more methods selected from the techniques such as a pH treatment, microfiltration, centrifugation and electrophoresis can be combined.

The present invention will now be described in more detail by way of Examples.

### EXAMPLES

“Preparation of Peptides”

To 5% by weight solution of soybean protein isolate (“FUJIPRO R” manufactured by FUJI OIL CO., LTD.), 2% by weight of Thermoase (origin; Bacillus thermoproteolyticus, Daiwa Fine Chemicals) per protein was added and reacted at pH 9.0 at 58° C. for 60 minutes. Next, to the obtained mixture, 1% by weight of Bioprase (origin; Bacillus sp., Nagase ChemteX Corporation) per protein was added and reacted at pH 7.5 at 58° C. for 60 minutes. To the obtained mixture, 1% by weight of Sumizyme FP (origin; Aspergillus sp., Shin Nihon Chemical Co., Ltd.) per protein was added and reacted at pH 7.5 at 58° C. for 60 minutes. After the above-described treatments, the reaction was stopped by heating at 85° C. for 20 minutes.

After the above treatments, resulting insoluble matters were removed by centrifugation and filtration with a membrane filter of 1.0 μm pore diameter to obtain “Peptide Composition 1”. In the molecular weight distribution of the peptide composition measured by a gel filtration method, the proportion of fractions having a molecular weight of 500 or less except for free amino acids was 45% by weight and the content of free amino acids was 5% by weight.

In addition, the content of the tripeptide expressed by phenylalanine-leucine-valine in Peptide Composition 1 was measured by HPLC with an ODS column and a PAD detector, and the result was 3% by weight.

As a comparative control, to the above-described 5% by weight solution of soybean protein isolate, 4% by weight of Bioprase (origin; Bacillus sp., Nagase ChemteX Corporation) per protein was added and reacted at pH 7.5 at 58° C. for 60 minutes. After the above treatment, the reaction was stopped by heating at 85° C. for 20 minutes. Then, resulting insoluble matters were removed by centrifugation and filtration with a membrane filter of 1.0 μm pore diameter to obtain “Peptide Composition 2”. In the molecular weight distribution of the peptide composition measured by a gel filtration method, the proportion of fractions having a molecular weight of 500 or less except for free amino acids was 21% by weight and the content of free amino acids was 1% by weight. The constitution of each peptide is shown in Table 2.

TABLE 2
Constitution of prepared peptide compositions

| | | | | | Free |
|---|---|---|---|---|---|
| Abundance of each fraction substance (% by weight) | | | | | amino |
| | A: | B: | C: | D: | acid (% by |
| | Molecular | Molecular | Molecular | Molecular | weight in |
| | weight | weight | weight | weight | the whole |
| | ≧12000 | 12000-500 | 500-240 | ≦240 | amount) |
| Peptide | 2 | 48 | 12 | 38 | 5 |
| Composition | | | | | |
| 1 | | | | | |
| Peptide | 10 | 68 | 7 | 15 | 1 |
| Composition | | | | | |
| 2 | | | | | |

### Example 1 and Comparative Examples 1 and 2

### Prevention and Treatment Test of Ulcerative Colitis

First, between 3 and 5 day-old 18 pigs were divided into 3 groups and they were bred for 3 days. Then the groups were designated as test groups (Example 1 and Comparative Example 1) and a control group (Comparative Example 2) and 1.25 g of dextran sulfate per kg of body weight was given as drinking water for 5 days (DSS treatment). Then, 250 mg of Peptide Composition 1 per kg of body weight was fed for 5 days in Example 1, 250 mg of Peptide Composition 2 per kg of body weight was fed for 5 days in Comparative Example 1, and alanine corresponding to the nitrogen content of the peptide was fed in Comparative Example 2. After that, the intestinal tract cells were collected during dissection and cytokines were measured. Comparative Example 2 corresponds to the control. (Dextran sulfate is a substance which induces bowel inflammation. International J. of Colorectal Disease, 15, 144-160 (2000))

“Measurement of Cytokines by an ELISA Method”

TNF-α and IL-6 were measured using a porcine ELISA kit (R&D system Inc. USA).

“Measurement of Cytokines by a RT-PCR Method”

As measurement of IL-17, mRNA was extracted by the RNA Mini Kit (Bio-Rad Lab. Inc.), and then cDNA was synthesized by the cDNA synthesizing kit (Bio-Rad Lab. Inc.), followed by measuring IL-17 using the SYBR Green Spermix (Bio-Rad Lab. Inc.).

“Identification of Absorbed Peptides”

Blood was collected during dissection and plasma was isolated, and then the structural analysis of a peak obtained by PDA-HPLC was carried out by LC-MS/MS to identify an amino acid sequence of the peptides.

“Statistical Significance Test”

Using the GraphPad Software (USA), the Tukey-Kramer comparison test was used for a statistical significance test.

“Test Results”

Analyses of TNF-α, IL-6 and IL-17

The analysis results of TNF-α, IL-6 and IL-17 are each shown in FIGS. 2 to 4. When the secretion amount of TNF-a in Comparative Example 2 was regarded as 100%, the secretion amount of TNF-α was 96% in Comparative Example 1 and 60% in Example 1. When the secretion amount of IL-6 in Comparative Example 2 was regarded as 100%, the secretion amount of IL-6 was 113% in Comparative Example 1 and 43% in Example 1. When the secretion amount of IL-17 in Comparative Example 2 was regarded as 100%, the secretion amount of IL-17 was 86% in Comparative Example 1 and 9% in Example 1. From the above, Example 1 was evaluated as effective because the secretion amounts of TNF-α, IL-6 and IL-17 in Example 1 were inhibited by 20% or more as compared to those of Comparative Example 2.

Statistical Significance Test

As a result of comparison with Comparative Example 2 by the Tukey-Kramer comparison test, Example 1 was evaluated as significant (risk rate: 5% or less). Comparative Example 1 was not evaluated as significant.

Analyses of Peptides in Plasma

The 3 peptide peaks were present in plasma in Example 1. The structural analysis of the largest peak of them was performed by LC-MS/MS to identify an amino acid sequence of the peptide. As a result, it was a tripeptide having an amino acid sequence, phenylalanine-leucine-valine.

In addition, an amino acid sequence of a peptide of the second largest peak was identified using the same method. As a result, it was a tripeptide having an amino acid sequence, valine-proline-tyrosine.

Analyses of Tissues

The micrographs of large intestinal tissue samples from pigs fed samples in Example 1 and Comparative Example 2 are shown in FIGS. 6 and 5. As shown in FIG. 6, few ulcers were confirmed in Example 1, while severe ulcers were confirmed in Comparative Example 2 of FIG. 5. (In Comparative Example 1, severe ulcers similar to those of Comparative Example 2 were confirmed).

“Inhibition Experiments of Inflammatory Cytokines by Cell Experiments Using Synthetic Peptides”

Syntheses of FLV and VPY

The synthesis of FLV was performed by a chemical technique in organic synthesis using phenylalanine, leucine and valine as raw materials and a carbodiimide condensing agent as a catalyst.

The synthesis of VPY was performed by a chemical technique in organic synthesis using valine, proline and tyrosine as raw materials and a carbodiimide condensing agent as a catalyst.

Examination of Inhibition of an Inflammatory Cytokine By Cell Experiments

Cultured Caco-2 cells were dispersed in 5% FBS-DMEM/F12 medium containing 3 mM FLV or VPY, and cultured for 2 hours. Then TNF-α was added to the medium, and the culture was performed for 4 hours to make an inflammatory condition. After a total of 6 hours, the supernatant was collected and once frozen at −80° C. After the frozen supernatant was thawed, an analysis of an inflammatory cytokine, IL-8, was performed by a normal ELISA method. The operation was repeated three times and a mean value was calculated and evaluated.

The results are shown in Table 3.

TABLE 3

| | Control | | | |
|---|---|---|---|---|
| | (only | TNF-α + | TNF-α + | TNF-α + |
| | Caco-2) | Caco-2 | Caco-2 + FLV | Caco-2 + VPY |
| IL-8 | 65.4 | 359.7 | 155.29 | — |
| concentration | | | | |
| (pg/ml) | | | | |
| IL-8 | 51.5 | 359.9 | — | 140.55 |
| concentration | | | | |
| (pg/ml) | | | | |

As shown in the above, it was found that the synthesized tripeptides FLV and VPY inhibit secretion of the inflammatory cytokine, IL-8, which is induced by TNF-α.

“Discussion”
In pigs to which Peptide Composition 1 was administered as in Example 1, it was confirmed by a tissue analysis that occurrences of bowel ulcers were inhibited or bowel ulcers were cured. Similarly, in pigs to which Peptide Composition 2 was administered (Comparative Example 1), bowel ulcers similar to those of Comparative Example 2 were confirmed. From these results, it was confirmed that a component having an effect of inhibiting the occurrences of bowel ulcers or curing bowel ulcers was present in Peptide Composition 1.
From the analysis of porcine plasma in Example 1, it was confirmed that secretion of TNF-α, IL-6 and IL-17 was inhibited, and that occurrences of pig bowel ulcers was inhibited or bowel ulcers were cured by a mechanism of inhibiting inflammation currently supposed.
In the present invention, the experiments were performed using pig bowel inflammation as a model. However, inhibition of inflammation and secretion behavior of TNF-α, IL-6 and IL-17 on this occasion were the same as those of a general anti-inflammatory action. Thus, the action of inhibiting bowel inflammation confirmed in the present experiments can be applied to the other general anti-inflammation.
The existence of three peptides was confirmed by analyzing peptides existing in plasma in Example 1, and the peptides were assumed to be active ingredients of anti-inflammation. A tripeptide having an amino acid sequence, phenylalanine-leucine-valine, was confirmed by analyzing an amino acid composition of one of the peptides, and the tripeptide was assumed to be an active ingredient of anti-inflammation.

A tripeptide having an amino acid sequence, valine-proline-tyrosine, was confirmed by analyzing the composition of another peptide of the above-described three peptides, and the tripeptide was also assumed to be an active ingredient of anti-inflammation.
In experiments of inhibiting an inflammatory cytokine by cell experiments using synthetic peptides FLV and VPY, FLV and VPY inhibited secretion of an inflammatory cytokine, IL-8, which was induced by TNF-α. This result strongly supports the results of Example 1.

### INDUSTRIAL APPLICABILITY

According to the present invention, there are provided oral anti-inflammatory compositions, foods, medicines and feedstuffs, which are safe, effective against inflammatory diseases such as inflammatory bowel disease and inhibit inflammatory cytokines of TNF-α, IL-6 and IL-17. In addition, according to the present invention, there are provided oral anti-inflammatory compositions, foods, medicines and feedstuffs, which contain a safe food material, which is effective against inflammatory diseases such as inflammatory bowel disease and inhibits inflammatory cytokines of TNF-α, IL-6 and IL-17, as an active ingredient.

### BRIEF DESCRIPTION OF DRAWINGS

FIG. 1 is a calibration curve using a molecular weight standard substance in determination of molecular weight.

FIG. 2 is a graph showing secretory behavior of TNF-a in Example 1 and Comparative Examples 1 and 2. The secretion of TNF-α was significantly inhibited in Example 1.

FIG. 3 is a graph showing secretory behavior of IL-6 in Example 1 and Comparative Examples 1 and 2. The secretion of IL-6 was significantly inhibited in Example 1.

FIG. 4 is a graph showing secretory behavior of IL-17 in Example 1 and Comparative Examples 1 and 2. The secretion of IL-17 was significantly inhibited in Example 1.

FIG. 5 is a micrograph of a large intestinal tissue sample of a pig in Comparative Example 2. In Comparative Example 1, severe bowel inflammation similar to that of Comparative Example 2 was confirmed.

FIG. 6 is a micrograph of a large intestinal tissue sample of a pig in Example 1. In Example 1, inflammation was not confirmed.

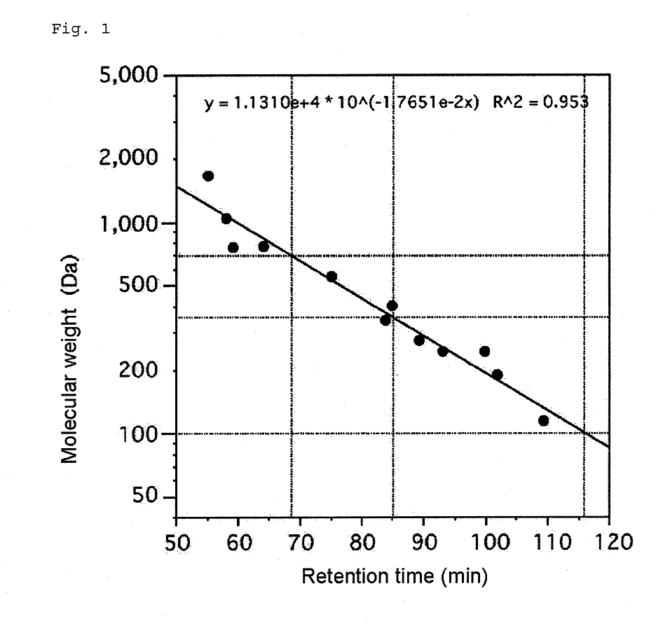

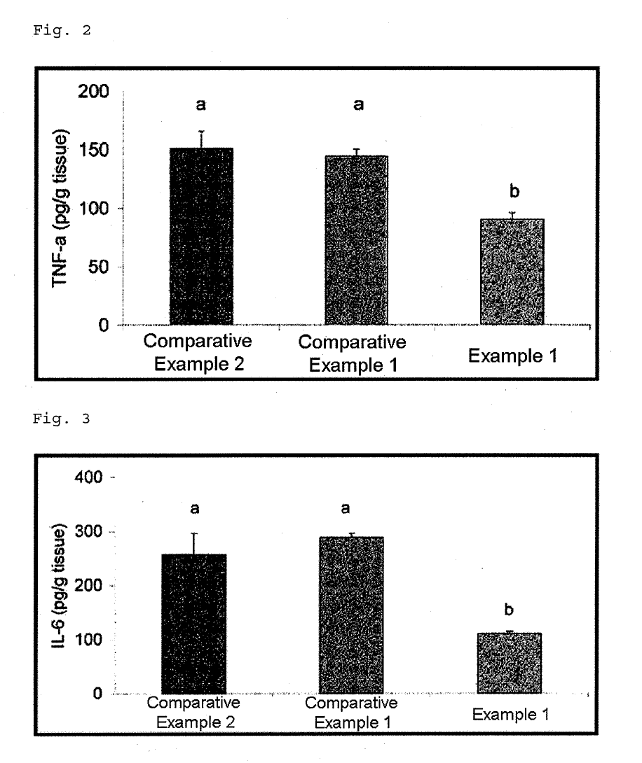

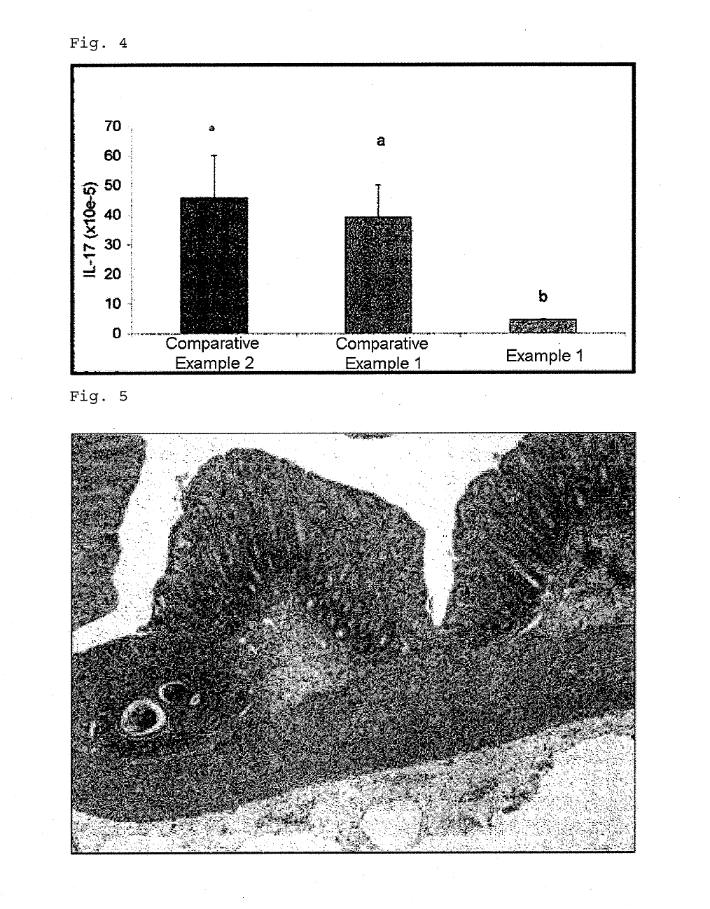

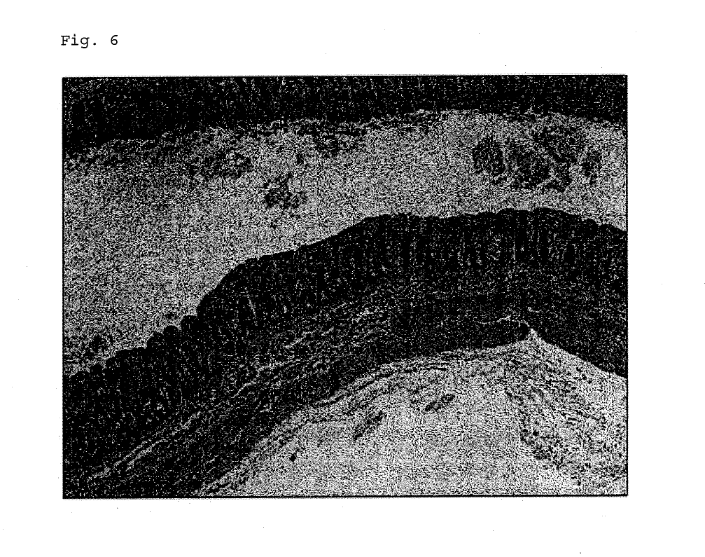

1-8. (canceled)

9. An oral anti-inflammatory functional agent comprising a soybean peptide, wherein the soybean peptide comprises 40% by weight or more of fractions having a molecular weight of 500 or less except for free amino acids, and wherein the soybean peptide comprises 7% by weight or less of the free amino acids.

10. The oral anti-inflammatory functional agent according to claim 9, wherein an intended disease of the agent is bowel inflammation.

11. A tripeptide which has an amino acid sequence Phe-Leu-Val or Val-Pro-Tyr.

12. An oral anti-inflammatory functional agent comprising the tripeptide according to claim 11 as an active ingredient.

13. An anti-secretory agent of inflammatory cytokines comprising the tripeptide according to claim 11 as an active ingredient.

14. A feedstuff comprising the tripeptide according to claim 11.

15. The oral anti-inflammatory functional agent according to claim 12, wherein an intended disease of the agent is bowel inflammation.

16. The anti-inflammatory functional agent according to claim 11, wherein the tripeptide is the tripeptide which has an amino acid sequence Phe-Leu-Val.

17. The anti-inflammatory functional agent according to claim 11, wherein the tripeptide is the tripeptide which has an amino acid sequence Val-Pro-Tyr.

18. A method of treating an inflammation, which comprises orally administering a peptide composition selected from a group consisting of a soybean peptide, a tripeptide which has an amino acid sequence Phe-Leu-Val, a tripeptide which has an amino acid sequence Val-Pro-Tyr and a combination thereof, wherein the soybean peptide comprises 40% by weight or more of fractions having a molecular weight of 500 or less except for free amino acids, and wherein the soybean peptide comprises 7% by weight or less of the free amino acids.

19. The method of treating an inflammation according to claim 18, wherein the peptide composition is the soybean peptide.

20. The method of treating an inflammation according to claim 18, wherein the peptide composition is the tripeptide which has an amino acid sequence Phe-Leu-Val.

21. The method of treating an inflammation according to claim 18, wherein the peptide composition is the tripeptide which has an amino acid sequence Val-Pro-Tyr.
